SEQUENCE LISTING (17 sequences)

SEQ ID NO: 1 - protein, 318 aa, Pichia stipitis
MPSIKLNSGYDMPAVGFGCWKVDVDTCSEQIYRAIKTGYRLFDGAEDYANEKLVGAGVKKAIDEGIVKREDLFLTSKLWNNYHHPDNVEKALNRTLSDLQVDYVDLFLIHFPVTFKFVPLEEKYPPGFYCGKGDNFDYEDVPILETWKALEKLVKAGKIRSIGVSNFPGALLLDLLRGATIKPSVLQVEHHPYLQQPRLIEFAQSRGIAVTAYSSFGPQSFVELNQGRALNTSPLFENETIKAIAAKHGKSPAQVLLRWSSQRGIAIIPKSNTVPRLLENKDVNSFDLDEQDFADIAKLDINLRFNDPWDWDKIPIFV

SEQ ID NO: 2 - protein, 363 aa, Pichia stipitis
MTANPSLVLNKIDDISFETYDAPEISEPTDVLVQVKKTGICGSDIHFYAHGRIGNFVLTKPMVLGHESAGTVVQVGKGVTSLKVGDNVAIEPGIPSRFSDEYKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVDIFDNKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPE

SEQ ID NO: 3 - protein, 600 aa, Saccharomyces cerevisiae
MLCSVIQRQTREVSNTMSLDSYYLGFDLSTQQLKCLAINQDLKIVHSETVEFEKDLPHYHTKKGVYIHGDTIECPVAMWLEALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILVGHLVELEEADACGMNLYDIRERKFSDELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWTLFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTFFVGGASKNDAIVKKFAQVIGATKGNFRLETPNSCALGGCYKAMWSLLYDSNKIAVPFDKFLNDNFPWHVMESISDVDNENWDRYNSKIVPLSELEKTLI

SEQ ID NO: 4 - DNA, 34 bp, Artificial Sequence
  Primer
ctagctagcaaagatgccgatttgggcgcgaatc

SEQ ID NO: 5 - DNA, 35 bp, Artificial Sequence
  Primer
acatgcatgcgtcgaccagctttaacgaacgcaga

SEQ ID NO: 6 - DNA, 34 bp, Artificial Sequence
  Primer
cccaagcttaaagatgccgatttgggcgcgaatc

SEQ ID NO: 7 - DNA, 34 bp, Artificial Sequence
  Primer
ctagctagcgtcgaccagctttaacgaacgcaga

SEQ ID NO: 8 - DNA, 33 bp, Artificial Sequence
  Primer
tccccccggggacagcttatcatcgataagctt

SEQ ID NO: 9 - DNA, 19 bp, Artificial Sequence
  Primer
cgcaaggaatggtgcatgc

SEQ ID NO: 10 - DNA, 32 bp, Artificial Sequence
  Primer
cggaattcatgttgtgttcagtaattcagaga

SEQ ID NO: 11 - DNA, 28 bp, Artificial Sequence
  Primer
gcggatccttagatgagagtcttttcca

SEQ ID NO: 12 - DNA, 34 bp, Artificial Sequence
  Primer
gacaggcgccaaagatgccgatttgggcgcgaat

SEQ ID NO: 13 - DNA, 34 bp, Artificial Sequence
  Primer
tccccccggggtcgaccagctttaacgaacgcag

SEQ ID NO: 14 - DNA, 30 bp, Artificial Sequence
  Primer
ggactagtagcatcttagtgaaaagggtgg

SEQ ID NO: 15 - DNA, 32 bp, Artificial Sequence
  Primer
ccgctcgagcgtaaggagaaaataccgcatca

SEQ ID NO: 16 - DNA, 34 bp, Artificial Sequence
  Primer
tccccgcggtaccacagcttttcaattcaattca

SEQ ID NO: 17 - DNA, 34 bp, Artificial Sequence
  Primer
tccccgcggtagggtaataactgatataattaaa

(19) United States
(12) Patent Application Publication
(10) Pub. No.: US 2012/0329104 A1
(43) Pub. Date: Dec. 27, 2012

(54) MODIFIED MICROORGANISM HAVING ENHANCED XYLOSE UTILIZATION

(75) Inventors: Jae Young KIM, Suwon-si (KR); Hyun Min KOO, Seoul (KR); Young kyoung PARK, Yongin-si (KR); Jae Chan PARK, Yongin-si (KR); Sung Min PARK, Yongin-si (KR); Jin Ho SEO, Seoul (KR); Yong Cheol PARK, Seoul (KR)

(73) Assignees: SEOUL UNIVERSITY RESEARCH AND BUSINESS FOUNDATION, Seoul (KR); SAMSUNG ELECTRONICS CO., LTD., Suwon-si (KR)

(21) Appl. No.: 13/535,016

(22) Filed: Jun. 27, 2012

(30) Foreign Application Priority Data
Jun. 27, 2011 (KR) 10-2011-0062316

(51) Int. Cl. C12N 1/19; C12N 1/15; C12N 15/63; C12N 1/21

(52) U.S. Cl. 435/106; 435/254.2; 435/252.3; 435/252.33; 435/252.34; 435/254.23; 435/254.22; 435/254.21; 435/252.32; 435/254.3; 435/254.11; 435/254.8; 435/252.31; 435/252.2; 435/252.35; 435/320.1; 435/136; 435/155; 435/161

(57) ABSTRACT

A modified microorganism having enhanced xylose utilization, an expression vector for constructing the modified microorganism, and a method of producing a chemical using the same are disclosed.

### CROSS-REFERENCE TO RELATED APPLICATION

This application claims priority to Korean Patent Application No. 10-2011-0062316, filed on Jun. 27, 2011, and all the benefits accruing therefrom under 35 U.S.C. §119, the content of which in its entirety is herein incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 14,007 Byte ASCII (Text) file named “709809_ST25.txt,” created on Jun. 27, 2012.

### BACKGROUND

1. Field

The disclosure relates to a modified microorganism having enhanced xylose utilization, an expression vector for constructing the modified microorganism, and a method of producing a chemical using the same.

2. Description of the Related Art

With globally increasing concern about the exhaustion of resources and pollution of the environment by overuse of fossil fuels, the production of a chemical using a microorganism is being considered. Cellulosic biomass is abundant in nature and is able to be cheaply harvested, thus it is being considered as a practical resource for producing the chemical.

However, the cellulosic biomass has not been widely employed in the industrial scale process for producing the chemical, due to absence of microorganisms capable of effectively converting the large amount of xylose which is contained in hydrolysates of the cellulosic biomass. Xylose accounts for about 25% by weight in metabolites of the cellulosic biomass. It is desirable to develop a microorganism capable of effectively converting xylose.

Currently, Saccharomyces cerevisiae (S. cerevisiae) is the most widely used microorganism used to produce a chemical from cellulosic biomass. However, the temperature suitable for growing S. cerevisiae should not be higher than a temperature of 35° C., and the ability of S. cerevisiae to utilize a carbon source including a pentose is low, thereby incurring a high cost in producing a chemical.

Recently, strains of Kluyveromyces have become attractive as a viable alternative to Saccharomyces cerevisiae. Kluyveromyces marxianus and Kluyveromyces Lactis are classified as GRAS (“Generally Recognized As Safe”), and may therefore be used with the same security as Saccharomyces cerevisiae.

K. marxianus is reported to grow at a temperature of 47° C., 49° C., or even 52° C., and to utilize a pentose such as xylose and arabinose as well as a polysaccharide such as lactose, inulin and cellobiose with an excellent ability.

However, a cofactor imbalance can arise from a metabolic pathway of xylose in K. marxianus, and xylitol, a byproduct of the xylose metabolism, is accumulated while production of a desired end product, i.e., ethanol, is low. Therefore, industrial use of xylose has been limited.

### SUMMARY

A modified microorganism including a biosynthetic pathway for effectively converting xylose derived from a cellulosic biomass to a chemical product is provided.

In one aspect, a modified microorganism comprising an activity of converting xylose to xylitol, an activity of converting xylitol to xylulose, and an activity of converting xylulose to xylulose-5-phosphate is provided.

In another aspect, an expression vector comprising a replication origin, a promoter, a gene encoding an activity of converting xylose to xylitol, a gene encoding an activity of converting xylitol to xylulose, and a gene encoding an activity of converting xylulose to xylulose-5-phosphate, and a terminator is provided.

In another aspect, the invention provides a method of producing a chemical comprising culturing a modified microorganism as disclosed herein in a xylose-containing medium, and recovering the chemical from the medium.

Desirably, the modified microorganism produces the chemical at a level greater than that produced by the precursor microorganism.

### BRIEF DESCRIPTION OF THE DRAWINGS

The above and other aspects of this disclosure will become more readily apparent by describing in further detail non-limiting exemplary embodiments thereof with reference to the accompanying drawings, in which:

FIG. 1 is a metabolic pathway of xylose.

FIG. 2 is a diagram depicting an expression vector according to Example 2.

### REFERENCE TO DEPOSITED BIOLOGICAL MATERIALS

Recombinant microorganisms in accordance with the compositions and methods described herein have deposited with the Korean Collection for Type Culture under accession numbers KCTC11951BP (Kluyveromyces marxianus (KCTC7155)/pKM316-_XRXDHXK_URA3), KCTC11952BP (Kluyveromyces marxianus (KCTC17555)/pKM316-_XRXDHXK_URA3), and KCTC11953BP (Kluyveromyces marxianus (KCTC17724)/pKM316-_XRXDHXK_URA3), each of which is considered to be an additional aspect of the invention.

### DETAILED DESCRIPTION

Unless otherwise indicated, the practice of the disclosure involves conventional techniques commonly used in molecular biology, microbiology, protein purification, protein engineering, protein and DNA sequencing, and recombinant DNA fields, which are within the skill of the art. Such techniques are known to those of skill in the art and are described in numerous standard texts and reference works. All patents, patent applications, articles and publications mentioned herein, both supra and infra, are hereby expressly incorporated herein by reference.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the Specification as a whole. It is to be understood that this disclosure is not limited to the particular methodology, protocols, and reagents described, as these may vary, depending upon the context they are used by those of skill in the art.

As used herein, the singular terms “a”, “an,” and “the” include the plural reference unless the context clearly indicates otherwise. Unless otherwise indicated, nucleic acids are written left to right in 5′ to 3′ orientation and amino acid sequences are written left to right in amino to carboxyl orientation, respectively.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not limitations of the various aspects or embodiments of the invention which can be had by reference to the specification as a whole.

Xylose Metabolism

A modified microorganism including a biosynthetic pathway for effectively converting xylose derived from a cellulosic biomass is provided.

As used interchangeably herein, the terms “biosynthetic pathway” or “metabolic pathway” refers to one or more (a set of) anabolic or catabolic biochemical reactions for converting one chemical species into another. Gene products belong to the same “metabolic pathway” if they, in parallel or in series, act on the same substrate, produce the same product, or act on or produce a metabolic intermediate between the same substrate and metabolite end product.

The modified microorganism may use a metabolic pathway of xylose as a biosynthetic pathway.

Xylose is a five-carbon monosaccharide that can be metabolized into useful products by a variety of organisms. Xylose is thought to be metabolized through a pentose phosphate pathway (“PPP”) after two other pathways of xylose metabolism are completed.

One pathway is called the “Xylose Reductase-Xylitol Dehydrogenase” or XR-XDH pathway. Xylose is converted to xylulose by the XR-XDH pathway. For example, xylose is reduced to xylitol by XR (“Xylose Reductase”) which is aided by cofactors NADH or NADPH, and xylitol is then oxidized to xylulose by XDH (“Xylitol Dehydrogenase”) which depends on the cofactor NAD⁺.

The other pathway for xylose metabolism is called the “Xylulokinase (XK)” pathway. Xylulose produced by the XR-XDH pathway is phosphorylated into xylulose-5-phosphate by XK, and then it may enter the pentose phosphate pathway for further catabolism.

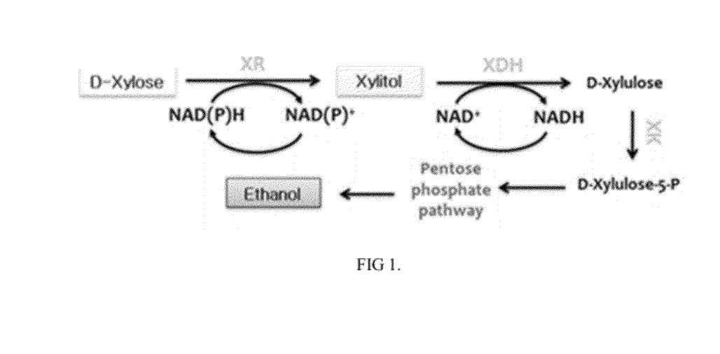

The metabolic pathway of xylose is shown in FIG. 1. Referring to FIG. 1, a cofactor imbalance arises from the fact that the XR reaction properly uses NADPH or NADH to produce NADP+ or NAD+, while the XDH reaction exclusively uses NAD+ and produces NADH. When less NADH is used in the XR reaction, less NAD⁺ is available for the XDH reaction. Therefore, the amount of NAD⁺ is insufficient, and xylitol is accumulated. In an exemplary embodiment, a modified microorganism capable of metabolizing xylitol is constructed.

As used herein, the term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a modified microorganism.

The xylose may be derived from a cellulosic biomass. As used herein, the terms “cellulosic biomass”, “lignocellulosic material”, and “lignocellulosic substrate” refer to any type of biomass comprising cellulose, hemicellulose, lignin, or combinations thereof. It may be derived from woody biomass, forage grasses, herbaceous energy crops, non-woody-plant biomass, agricultural wastes, agricultural residues, forestry residues, forestry wastes, paper-production sludge, waste paper sludge, waste-water-treatment sludge, municipal solid waste, corn fiber from wet and dry mill corn ethanol plants, and sugar-processing residues, but is not limited thereto.

The woody biomass may include recycled wood pulp fiber, sawdust, hardwood and softwood, the grasses may include switch grass, cord grass, rye grass, reed canary grass and miscanthus, the sugar-processing residues may include sugar cane bagasse, the agricultural wastes may include rice straw, rice hulls, barley straw, corn cobs, cereal straw, wheat straw, canola straw, oat straw, oat hulls and corn fiber, the stover may include soybean stover and corn stover, the forestry wastes may include recycled wood pulp fiber, sawdust, hardwood and softwood, but is not limited thereto. The lignocellulosic material may comprise one species of fiber, or the lignocellulosic material may comprise a mixture of fibers that originate from different lignocellulosic materials.

Modified Microorganism

In an aspect, a modified microorganism comprising an activity of converting xylose to xylitol, an activity of converting xylitol to xylulose, and an activity of converting xylulose to xylulose-5-phosphate is provided.

As used herein, the term “metabolically engineered” or “metabolic engineering” involves rational pathway design and assembly of biosynthetic genes, genes associated with operons, and control elements of such nucleic acid sequences, for the production of a desired metabolite, such as an alcohol, in a microorganism. “Metabolically engineered” can further include optimization of metabolic flux by regulation and optimization of transcription, translation, protein stability and protein functionality using genetic engineering and appropriate culture condition. The biosynthetic genes can be heterologous to the host (e.g., microorganism), either by virtue of being foreign to the host, or being modified by mutagenesis, recombination, or association with a heterologous expression control sequence in an endogenous host cell. Appropriate culture conditions are conditions such as culture medium pH, ionic strength, nutritive content, etc., temperature, oxygen, CO₂, nitrogen content, humidity, and other culture conditions that permit production of the compound by the host microorganism, i.e., by the metabolic action of the microorganism. Appropriate culture conditions are well known for microorganisms that can serve as host cells.

Accordingly, a metabolically “engineered” or “modified” microorganism, which can also be called a “recombinant” microorganism, is produced via the introduction of genetic material into a host or parental microorganism of choice thereby modifying or altering the cellular physiology and biochemistry of the microorganism. Through the introduction of genetic material the parental microorganism acquires new properties, e.g. the ability to produce a new, or greater quantities of, an intracellular metabolite.

For example, the introduction of genetic material into a parental microorganism results in a new or modified ability to produce a chemical. The genetic material introduced into the parental microorganism contains one or more genes, or parts of genes, coding for one or more of the enzymes involved in a biosynthetic pathway for the production of a chemical and may also include additional elements for the expression or regulation of expression of these genes, e.g. promoter sequences. In the embodiment, a microorganism may be modified to have an activity for the conversion of xylose to xylulose-5-phosphate.

As used interchangeably herein, the terms “activity” and “enzymatic activity” refer to any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof.

The activity of converting xylose to xylitol indicates the ability to reduce xylose into xylitol with NADPH or NADH as a cofactor.

The activity of converting xylose to xylitol may be a xylose reductase (“XR”) activity. The XR activity may be derived from xylose-utilizing yeasts such as Candida shetatae, Pichia stipitis and Pachysolen tannophilus, but is not limited thereto. In other words, a xylose reductase enzyme, or nucleic acid encoding such enzyme, from a xylose-utilizing yeast can be used to provide the xylose reductase activity. In an exemplary embodiment, the XR activity (XR enzyme or nucleic acid encoding same) from Pichia stipitis is used, which has the amino acid sequence provided as SEQ ID NO: 1.

SEQ ID NO: 1
M P S I K L N S G Y D M P A V G F G C W K V D V D
T C S E Q I Y R A I K T G Y R L F D G A E D Y A N
E K L V G A G V K K A I D E G I V K R E D L F L T
S K L W N N Y H H P D N V E K A L N R T L S D L Q
V D Y V D L F L I H F P V T F K F V P L E E K Y P
P G F Y C G K G D N F D Y E D V P I L E T W K A L
E K L V K A G K I R S I G V S N F P G A L L L D L
L R G A T I K P S V L Q V E H H P Y L Q Q P R L I
E F A Q S R G I A V T A Y S S F G P Q S F V E L N
Q G R A L N T S P L F E N E T I K A I A A K H G K
S P A Q V L L R W S S Q R G I A I I P K S N T V P
R L L E N K D V N S F D L D E Q D F A D I A K L D
I N L R F N D P W D W D K I P I F V

The activity of converting xylitol to xylulose indicates the ability to oxidize xylitol into xylulose with NAD⁺ as a cofactor.

The activity of converting xylitol to xylulose may be a xylitol dehydrogenase (“XDH”) activity. The XDH activity may be derived from xylose-utilizing yeasts such as Candida shetatae, Pichia stipitis and Pachysolen tannophilus, but not is limited thereto. In other words, a xylose reductase enzyme, or nucleic acid encoding such enzyme, from a xylose-utilizing yeast can be used to provide the XDH activity. In an exemplary embodiment, the XDH activity (XDH enzyme or nucleic acid encoding same) from Pichia stipitis is used, which has the amino acid sequence provided as SEQ ID NO: 2.

SEQ ID NO: 2
M T A N P S L V L N K I D D I S F E T Y D A P E I
S E P T D V L V Q V K K T G I C G S D I H F Y A H
G R I G N F V L T K P M V L G H E S A G T V V Q V
G K G V T S L K V G D N V A I E P G I P S R F S D
E Y K S G H Y N L C P H M A F A A T P N S K E G E
P N P P G T L C K Y F K S P E D F L V K L P D H V
S L E L G A L V E P L S V G V H A S K L G S V A F
G D Y V A V F G A G P V G L L A A A V A K T F G A
K G V I V V D I F D N K L K M A K D I G A A T H T
F N S K T G G S E E L I K A F G G N V P N V V L E
C T G A E P C I K L G V D A I A P G G R F V Q V G
N A A G P V S F P I T V F A M K E L T L F G S F R
Y G F N D Y K T A V G I F D T N Y Q N G R E N A P
I D F E Q L I T H R Y K F K D A I E A Y D L V R A
G K G A V K C L I D G P E

The activity of converting xylulose to xylulose-5-phosphate indicates the ability to convert phosphorylate xylulose into xylulose-5-phosphate with ATP (“Adenosine triphosphate”).

The activity of converting xylulose to xylulose-5-phosphate may be a xylulokinase (“XK”) activity. The XK activity may be derived from xylose-utilizing yeasts such as Candida shetatae, Pichia stipitis and Pachysolen tannophilus, as well as xylose non-utilizing yeasts such as Saccharomyces cerevisiae, Schizoxaccaromyces pombe and Escherichia coli, but is not limited thereto. In other words, a xyluokinase enzyme, or nucleic acid encoding such enzyme, from a xylose-utilizing yeast can be used to provide the XK activity. In an exemplary embodiment, the XK activity (e.g., enzyme or nucleic acid encoding same) from Saccharomyces cerevisiae is used, which has the amino acid sequence provided as SEQ ID NO: 3.

SEQ ID NO: 3
M L C S V I Q R Q T R E V S N T M S L D S Y Y L G
F D L S T Q Q L K C L A I N Q D L K I V H S E T V
E F E K D L P H Y H T K K G V Y I H G D T I E C P
V A M W L E A L D L V L S K Y R E A K F P L N K V
M A V S G S C Q Q H G S V Y W S S Q A E S L L E Q
L N K K P E K D L L H Y V S S V A F A R Q T A P N
W Q D H S T A K Q C Q E F E E C I G G P E K M A Q
L T G S R A H F R F T G P Q I L K I A Q L E P E A
Y E K T K T I S L V S N F L T S I L V G H L V E L
E E A D A C G M N L Y D I R E R K F S D E L L H L
I D S S S K D K T I R Q K L M R A P M K N L I A G
T I C K Y F I E K Y G F N T N C K V S P M T G D N
L A T I C S L P L R K N D V L V S L G T S T T V L
L V T D K Y H P S P N Y H L F I H P T L P N H Y M
G M I C Y C N G S L A R E R I R D E L N K E R E N
N Y E K T N D W T L F N Q A V L D D S E S S E N E
L G V Y F P L G E I V P S V K A I N K R V I F N P
K T G M I E R E V A K F K D K R H D A K N I V E S
Q A L S C R V R I S P L L S D S N A S S Q Q R L N
E D T I V K F D Y D E S P L R D Y L N K R P E R T
F F V G G A S K N D A I V K K F A Q V I G A T K G
N F R L E T P N S C A L G G C Y K A M W S L L Y D
S N K I A V P F D K F L N D N F P W H V M E S I S
D V D N E N W D R Y N S K I V P L S E L E K T L I

The activity of converting xylose to xylitol, the activity of converting xylitol to xylulose, and the activity of converting xylulose to xylulose-5-phosphate may be introduced to a microorganism by a known method in the art. For example, the method may include manufacturing a expression vector including a gene having the activities (e.g., encoding enzymes having the activities), and then transforming a microorganism with the expression vector.

Expression Vector

In another embodiment, an expression vector comprising a promoter; a gene encoding an activity of converting xylose to xylitol, a gene encoding an activity of converting xylitol to xylulose, and a gene encoding an activity of converting xylulose to xylulose-5-phosphate; and a terminator is provided.

As used herein, the term “expression vector” refers to a DNA construct containing a DNA sequence that is operably linked to a suitable control sequence capable of effecting the expression of the DNA in a suitable host. The vector may be a plasmid, a phage particle, or simply a potential genomic insert. Once transformed into a suitable host, the vector replicates and functions independently of the host genome, or integrates into the genome itself. As used herein, the terms “plasmid,” “expression plasmid,” and “vector” are often used interchangeably as the plasmid is the most commonly used form of vector at present.

However, it is intended to include such other forms of expression vectors that serve equivalent functions and which are, or become, known in the art. For example, the vector may be include cloning vectors, expression vectors, shuttle vectors, plasmids, phage or virus particles, DNA constructs, cassettes and the like. As used herein, the term “plasmid” refers to a circular double-stranded DNA construct used as a cloning vector, and which forms an extrachromosomal self-replicating genetic element in many bacteria and some eukaryotes. The plasmid may include multicopy plasmids that can integrate into the genome of the host cell by homologous recombination.

As known to those skilled in the art, in order to increase the expression level of a gene introduced to a host cell, the gene should be operably linked to expression control sequences for the control of transcription and translation which function in the selected expression host. For example, the expression control sequences and the gene are included in one expression vector together with a selection marker and a replication origin. When the expression host is a eukaryotic cell, the expression vector should further include an expression marker useful in the eukaryotic expression host.

As used herein, the term “operably linked” refers that elements are arranged to perform the general functions of the elements. A nucleic acid is said to be “operably linked” when it is placed into a functional relationship with another nucleic acid sequence. For example, a polynucleotide promoter sequence is operably linked to a polynucleotide encoding a polypeptide if it affects the transcription of the sequence. The term “operably linked” may mean that the polynucleotide sequences being linked are contiguous. Linking may be accomplished by ligation at convenient restriction sites. If such sites do not exist, synthetic oligonucleotide adaptors or linkers may be used in accordance with conventional practice.

As used herein, the term “promoter” refers to a nucleic acid sequence that functions to drive or effect the transcription of a downstream gene. The promoter may be any promoter that drives the expression of a target protein, and may be any nucleic acid sequence which shows transcriptional activity in the host cell of choice and includes mutant, truncated and hybrid promoters, and may be obtained from genes encoding extracellular or intracellular polypeptides either homologous or heterologous to the host cell. The promoter sequence may be native or foreign to the host cell.

As used herein, the term “gene” refers to a nucleotide sequence that encodes a gene product, such as a protein or enzyme, including a chromosomal or non-chromosomal segment of DNA involved in producing a polypeptide chain that may or may not include regions preceding and following the coding regions, for example, 5′ untranslated (“5′ UTR”) or leader sequences and 3′ untranslated (“3 UTR”) or trailer sequences, as well as intervening sequence (introns) between individual coding segments (exons).

As used interchangeably herein, the terms “polynucleotide” and “nucleic acid” refer to a polymeric form of nucleotides of any length. These terms include, but are not limited to, a single-stranded DNA (“deoxyribonucleic acid”), double-stranded DNA, genomic DNA, cDNA, or a polymer comprising purine and pyrimidine bases, or other natural, chemically, biochemically modified, non-natural or derivatized nucleotide bases. Non-limiting examples of polynucleotides include genes, gene fragments, chromosomal fragments, ESTs, exons, introns, mRNA, tRNA, rRNA, ribozymes, cDNA, recombinant polynucleotides, branched polynucleotides, plasmids, vectors, isolated DNA of any sequence, isolated RNA (“ribonucleic acid”) of any sequence, nucleic acid probes, and primers. It will be understood that, as a result of the degeneracy of the genetic code, a multitude of nucleotide sequences encoding a given protein may be produced.

As used herein, the term “terminator” refers to a nucleic acid sequence that functions to drive or effect termination of transcription.

The promoter may be selected from the group consisting of PGK (“phosphoglycerate kinase 1”), CYC (“cytochrome-c oxidase”), TEF (“translation elongation factor 1α”), GPD (“glyceraldehyde-3-phosphate dehydrogenase”), ADH (“alcohol dehydrogenase”), PHO5, TRP1, GAL1, GAL10, hexokinase, pyruvate decarboxylase, phosphofructokinase, triose phosphate isomerase, phosphoglucose isomerase, glucokinase, α-mating factor pheromone, GUT2, nmt, fbp1, AOX1, AOX2, MOX1 and FMD1, but is not limited thereto. In an exemplary embodiment, the PGK1 promoter is used.

The activity of converting xylose to xylitol is encoded by a nucleic acid encoding xylose reductase. Any nucleotide sequence encoding a xylose reductase can be used, such as an XYL1 gene. The XYL1 gene may comprise a nucleotide sequence encoding an enzyme represented by SEQ ID NO: 1, or at least about 70%, at least about 75%, at least about 80%, at least about 85%, at least about 90%, at least about 92%, at least about 95%, at least about 97%, at least about 98% or at least about 99% sequence homology with SEQ ID NO: 1.

The activity of converting xylitol to xylulose is encoded by a nucleic acid encoding a xylulose dehydrogenase. Any nucleotide sequence encoding xylulose dehydrogenase can be used, such as the XYL2 gene. The XYL2 gene may comprise a nucleotide sequence encoding an enzyme represented by SEQ ID NO: 2, or a nucleotide sequence having at least about 70%, at least about 75%, at least about 80%, at least about 85%, at least about 90%, at least about 92%, at least about 95%, at least about 97%, at least about 98% or at least about 99% sequence homology with SEQ ID NO: 2.

The activity of converting xylulose to xylulose-5-phosphate is encoded by a nucleic acid encoding a xyluokinase enzyme. Any nucleotide sequence encoding a xyluokinase enzyme can be used, such as the XKS1 gene. The XKS1 gene may comprise a nucleotide sequence encoding an enzyme represented by SEQ ID NO: 3, or a nucleotide sequence comprising at least about 70%, at least about 75%, at least about 80%, at least about 85%, at least about 90%, at least about 92%, at least about 95%, at least about 97%, at least about 98% or at least about 99% sequence homology with SEQ ID NO: 3.

As used herein, the term “homology” refers to sequence similarity or sequence identity. This homology or identity (e.g., percent identity) may be determined using standard techniques known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

The terminator may be selected from the group consisting of PGK1 (“phosphoglycerate kinase 1”), CYC1 (“Cytochrome c transcription”) and GAL1, but is not limited thereto. In an exemplary embodiment, the PGK1 terminator is used.

The expression vector may further comprise a selection marker. As used herein, the term “selection marker” refers to a nucleotide sequence which is capable of expression in the host cells and where expression of the selection marker confers to cells containing the expressed gene the ability to grow in the presence of a corresponding selective agent or lack of an essential nutrient. For example, the selection marker may include, but is not limited to, antimicrobials such as kanamycin, erythromycin, actinomycin, chloramphenicol and tetracycline, and auxotrophs such as URA3, LEU2, TRP1 and HIS3. That is, the selection markers are genes that confer antimicrobial resistance or an auxotroph on the host cell to allow cells containing the exogenous DNA to be distinguished from cells that have not received any exogenous sequence during the transformation. In an exemplary embodiment, the URA3 auxotroph is used as a selection marker.

The expression vector may further comprise a replication origin. As used herein, the term “replication origin” refers to a nucleotide sequence which begins a replication or an amplification of a plasmid in a host cell. The replication origin may include an autonomous replication sequence (“ARS”), and the ARS may be stabilized by a centromeric sequence (“CEN”). In an exemplary embodiment, ARS/CEN from Kluyveromyces marxianus is used.

Construction of Modified Microorganism

The expression vector may be introduced into a host cell by a known method in the art.

As used herein, the term “host cell” refers to a cell that serves as a host for an expression vector. A suitable host cell may be a naturally occurring or wild-type host cell, or it may be an altered host cell. A “wild-type host cell” is a host cell that has not been genetically altered using recombinant methods.

As used herein, the term “altered host cell” refers to a genetically engineered host cell wherein a target protein is expressed or produced at a level of expression or production that is greater than the level of expression or production of the same target protein in an unaltered or wild-type host cell grown under essentially the same growth conditions. A “modified host cell” herein refer to a wild-type or altered host cell that has been genetically engineered to express or overexpress a non-native or other target protein. The modified host cell is preferably capable of expressing a target protein at a greater level than its wild-type or altered parent host cell.

As used herein, the term “parent” or “precursor” cell refers to a cell from which a modified or recombinant host cell is derived. The parent or precursor cell can be a wild-type cell or an altered (e.g., recombinant) cell.

For example, the genus of available host cells may be one selected from the group consisting of Zymomonas, Escherichia, Pseudomonas, Alcaligenes, Salmonella, Shigella, Burkholderia, Oligotropha, Klebsiella, Pichia, Candida, Hansenula, Saccharomyces, Kluyveromyces, Comamonas, Corynebacterium, Brevibacterium, Rhodococcus, Azotobacter, Citrobacter, Enterobacter, Clostridium, Lactobacillus, Aspergillus, Zygosaccharomyces, Dunaliella, Debaryomyces, Mucor, Torulopsis, Methylobacteria, Bacillus, Rhizobium and Streptomyces.

In the embodiment, the host cell may be, but is not limited to, a cell from the genus Kluyveromyces or the genus Escherichia. For example, the genus Kluyveromyces may include K. marxianus, K. fragilis, K. lactis, K. bulgaricus, and K. thermotolerans, but is not limited thereto. In an exemplary embodiment, K. marxianus and E. coli are used.

As used herein, the term “introduced” refers to any method suitable for transferring the nucleic acid sequence into the cell, such that it can be expressed. Such method for introduction may includes, but be not limited to, protoplast fusion, transfection, transformation, conjugation, and transduction (See e.g., Ferrari et al., “Genetics,” in Hardwood et al., (eds.), Bacillus, Plenum Publishing Corp., pages 57-72, [1989]).

As used herein, the terms “transformed” and “stably transformed” refer to a cell that has a non-native heterologous polynucleotide sequence integrated into its genome or has the heterologous polynucleotide sequence present as an episomal plasmid that is maintained for at least two generations.

The introduction of the gene encoding the activity of converting xylose to xylitol, the gene encoding the activity of converting xylitol to xylulose, and the gene encoding the activity of converting xylulose to xylulose-5-phosphate to a host cell may be performed by isolating a plasmid from E. coli and then by transforming the plasmid into the host cell. However, it is not essential to use intervening microorganisms such as E. coli. A vector can be directly introduced into a host cell. Transformation may be achieved by any one of various means including electroporation, microinjection, biolistics (or particle bombardment-mediated delivery), or agrobacterium-mediated transformation.

Method of Producing Chemical

A method of producing a desired chemical also is provided, which method comprises culturing the modified microorganism in a xylose-containing medium, and recovering the chemical from the medium.

The step of culturing the modified microorganism may be performed under conditions suitable for the production of the chemical. The medium used to culture the cells comprises any conventional medium suitable for growing the host cells, such as minimal or complex media containing appropriate supplements. Suitable media are available from commercial suppliers or may be prepared according to published recipes (e.g., in catalogues of the American Type Culture Collection).

In one embodiment, the medium contains xylose as a carbon source. The xylose may be xylose itself, or an oligomeric or polymeric carbohydrate which comprises xylose units, such as lignocellulose, arabinan, cellulose, and starch. To release the xylose units from the carbohydrate, a suitable carbohydrate (e.g., xylanase) may be added to the medium.

The medium may further contain other carbon sources, such as glucose or blackstrap; nitrogen sources such as ammonia, ammonium sulfate, ammonium chloride, ammonium nitrate, or urea; inorganic salts such as potassium hydrogen phosphate, potassium dihydrogen phosphate, or magnesium sulfate; or any combination thereof. In addition, or instead, nutrients such as peptone, meat extract, yeast extract, corn steep liquor, casamino acids, and various vitamins, such as biotin and thiamine, may be added to the medium if needed.

The modified microorganism may be cultured under batch, fed-batch or continuous fermentation conditions. Classical batch fermentation methods use a closed system, wherein the culture medium is made prior to the beginning of the fermentation run, the medium is inoculated with the desired organisms, and fermentation occurs without the subsequent addition of any components to the medium. In certain cases, the pH and oxygen content of the growth medium, but not the carbon source content, are altered during batch methods. The metabolites and cell biomass of the batch system change constantly up to the time the fermentation is stopped. In a batch system, cells usually progress through a static lag phase to a high growth log phase and finally to a stationary phase where growth rate is diminished or halted. If untreated, cells in the stationary phase eventually die. Generally, cells produce the most protein in the log phase.

A variation on the standard batch system is the “fed-batch fermentation” system. In this system, nutrients (e.g., a carbon source, nitrogen source, O₂, and typically, other nutrients) are only added when their concentration in culture falls below a threshold. Fed-batch systems are useful when catabolite repression is apt to inhibit the metabolism of the cells, and where it is desirable to have limited amounts of nutrients in the medium. Measurement of the actual nutrient concentration in fed-batch systems is estimated on the basis of the changes of measurable factors such as pH, dissolved oxygen and the partial pressure of waste gases such as CO₂. Batch and fed-batch fermentations are common and well known in the art.

Continuous fermentation employs an open system in which a defined culture medium is added continuously to a bioreactor and an equal amount of conditioned medium is removed simultaneously for processing. Continuous fermentation generally maintains the cultures at a constant high density where cells are primarily in log-phase growth. Continuous fermentation allows for the modulation of one factor or any number of factors that affect cell growth and/or end product concentration. For example, a limiting nutrient, such as the carbon source or nitrogen source, is maintained at a fixed rate and all other parameters are allowed to moderate. In other systems, a number of factors affecting growth are altered continuously while the cell concentration, measured by media turbidity, is kept constant. Continuous systems strive to maintain steady state growth conditions. Thus, cell loss due to medium being drawn off may be balanced against the cell growth rate in the fermentation. Methods of modulating nutrients and growth factors for continuous fermentation processes as well as techniques for maximizing the rate of product formation are known to those of skill in the art.

The step of recovering the chemical from the medium may be performed by any suitable method. For example, the method may include salting-out, recrystallization, extraction with organic solvent, esterification distillation, chromatography, and electrodialysis, and the method for separation, purification, or collection may be appropriately selected according to the characteristics of the chemical.

In some embodiments, the chemical may comprise, consist essentially of, or consist of one or more organic acids, alcohols, amino acids, or vitamins, but is not limited thereto.

Organic acids which can be produced may include acetic acid, lactic acid, 3-hydroxypropionic acid, acrylic acid, succinic acid, fumaric acid, malic acid, oxalacetic acid, citric acid, cis-aconitic acid, isocitric acid, itaconic acid, 2-oxoglutaric acid and shikimic acid. The alcohol may include ethanol, butanol, 1,3-propanediol, glycerol, xylitol, sorbitol and 1,4-butanediol. The amino acid may include valine, leucine, alanine, aspartic acid, lysine, isoleucine and threonine.

Producing organic acids via xylose fermentation using a modified microorganism has been studied. Xylose may be one of the renewable biomasses for fermentation to industrially useful acetic acid. Acetic acid may be produced with a theoretical weight yield of 100% by the modified Clostridium thermoaceticum strain (ATCC 49707) using xylose as the carbon source. The production of optically pure d-lactic acid via xylose fermentation may be achieved by using a Lactobacillus plantarum strain (NCIMB 8826). Succinic acid may be produced via xylose fermentation in high yields. A metabolically engineered E. coli strain (AFP184), which is able to produce succinic acid by fermentation from both glucose and xylose feedstocks, may be used for succinic acid production.

In an exemplary embodiment, the chemical produced is ethanol.

In preferred embodiments, the production of the desired chemical in the modified microorganism may be increased as compared with that in the precursor microorganism.

As used herein, the term “increased” or “increasing” production of a product or molecule refers to the ability of one or more recombinant microorganisms to produce a greater amount of a given product or molecule for a given amount of source material (e.g., xylose) or over a given period of time as compared to a control microorganism, such as a precursor microorganism or a differently modified microorganism. An “increased” amount is typically a “statistically significant” amount, and may include an increase that is 10%, 20%, 30%, and 40% etc., than the amount produced by a precursor microorganism or a differently modified microorganism.

In an exemplary embodiment, the maximum yield of ethanol produced by the modified microorganism is about 6.6%. For example, about 0.066 g ethanol per gram of xylose is produced in KM8 strain. Desirably, the yield in the modified microorganism is at least about 125%, at least about 137%, at least about 160%, at least about 166%, or at least about 275%, or more than that of the precursor microorganism, allowing the chemical to be industrially produced in large quantities more easily.

Hereinafter, the invention will be described in further detail with respect to exemplary embodiments. However, it should be understood that the invention is not limited to these Examples and may be embodied in various modifications and changes.

### EXAMPLES

### Strain and Plasmid

E. coli TOP10 F-mcrA Δ(mrr-hsdRMS-mcrBC) φ80lacZΔM15 ΔlacX74 nupG recA1 araD139 Δ(ara-leu)7697 galE15 galK16 rpsL(StrR) endA1λ-(Invitrogen, CA) is used for amplification of a plasmid. The strain of Kluyveromyces marxianus var. marxianus, for example, KM3(KCTC 17555), KM8(KCTC4155), and KM11(KCTC17724) is used as a yeast host cell. pRS306 (ATCC 77141) is used as a plasmid for recombination of a gene.

Medium and Method for Culturing

E. coli is inoculated in LB medium (1% bacto-trypton, 0.5% bacto-yeast extract, 1% NaCl) having ampicillin and kanamycin, and then cultured at a temperature of 37° C. A yeast host cell and a recombinant yeast are cultured in YPD medium (1% bacto-yeast extract, 2% bacto-pepton, 2% dextrose) at a temperature of 37° C. for 2 days. Minimal medium includes 0.17% yeast nitrogen base, 0.5% ammonium sulfate, 2% glucose or glycerol, 38.4 mg/l arginine, 57.6 mg/l isoleucine, 48 mg/l phenylalanine, 57.6% mg/l valine, 6 mg/l threonine, 50 mg/l inositol, 40 mg/l tryptophan, 15 mg/l tyrosine, 60 mg/l leucine and 4 mg/l histidine.

### Example 1

The following example illustrates an expression analysis of K. marxianus.

The K. marxianus strains, KM3(KCTC 17555), KM8(KCTC 4155), and KM11(KCTC17724) are precultured overnight in LB medium, and inoculated in the same medium including additional 10% xylose so that an initial value of OD600 is 1.

About 10 ml of the culture in the 20 ml tube is placed in a rotary shaker at a 30 degree angle above the horizon, and cultured at 200 rpm for 3 days at 37° C. The amounts of xylose, xylitol, glycerol and ethanol which are contained in fermentation liquor are measured by means of HPLC, and then the result is shown in Table 1.

TABLE 1

| | ODmax | Xylose consumed | Xylitol | Glycerol | Ethanol | Yield x/x |
|---|---|---|---|---|---|---|
| Strains | (g/L) | (g/L) | (g/L) | (g/L) | (g/L) | (g xylitol/g xylose) |
| KM3 | 22.1 | 54.9 | 5.7 | 0 | 0 | 0.104 |
| (KCTC17555) | | | | | | |
| KM8 | 5.0 | 6.8 | 2.3 | 0 | 0 | 0.338 |
| (KCTC7155) | | | | | | |
| KM11 | 0.75 | ~0 | 0 | 0 | 0 | — |
| (KCTC17724) | | | | | | |

Referring to Table 1, it can be seen that K. marxianus strains do not produce ethanol due to the accumulation of xylitol which is a metabolic intermediate.

### Example 2

The following example illustrates the construction of an expression vector in accordance with the invention.

A. pYip5XR

PGK1 promoter and terminator from S. cerevisiae, and XYL1 gene from P. stipitis are amplified using YepM4-XR (Microbiology, Microbiology (2007), 153, 3044-3054) as a template by means of PCR at an optimal annealing temperature (TaOpt) of 55° C. The used primers are follows:

Forward(FW) primer:
(SEQ ID NO: 4)
5′-CTAGCTAGCAAAGATGCCGATTTGGGCGCGAATC-3′
Backward(BW) primer:
(SEQ ID NO: 5)
5′-ACATGCATGCGTCGACCAGCTTTAACGAACGCAGA-3′

Next, the gene is digested with restriction enzymes NheI and SphI, and then ligated into the plasmid Yip5(ATCC37061) which is digested with the same restriction enzyme to construct pYip5XR.

B. pYip5XRXDH

PGK1 promoter and terminator from S. cerevisiae, and XYL2 gene from P. stipitis are amplified using pPGK-XDH (Microbiology, Microbiology (2007), 153, 3044-3054) as a template by means of PCR at an optimal annealing temperature of 55° C. The used primers are follows:

FW primer:
(SEQ ID NO: 6)
5′-CCCAAGCTTAAAGATGCCGATTTGGGCGCGAATC-3′
BW primer:
(SEQ ID NO: 7)
5′-CTAGCTAGCGTCGACCAGCTTTAACGAACGCAGA-3′

Next, the gene is digested with restriction enzymes HindIII and NheI, and then ligated into the plasmid pYip5XR which is digested with the same restriction enzyme to construct pYip5XRXDH.

C. pAUR101_XRXDH

PGK1 promoter and terminator from S. cerevisiae, and XYL1 and XYL2 gene from P. stipitis are amplified using pYip5XRXDH as a template by means of PCR at an optimal annealing temperature of 55° C. The used primers are follows:

FW primer:
(SEQ ID NO: 8)
5′-TCCCCCCGGGGACAGCTTATCATCGATAAGCTT-3′
BW primer:
(SEQ ID NO: 9)
5′-CGCAAGGAATGGTGCATGC-3′

Next, the gene is digested with restriction enzymes XmaI and SphI, and then ligated into the plasmid pAUR101 which is digested with the same restriction enzyme to construct pAUR101_XRXDH.

D. pPGKXK

XKS1 gene from S. cerevisiae is amplified by means of PCR at an optimal annealing temperature of 55° C. The used primers are follows:

FW primer:
(SEQ ID NO: 10)
5′-CGGAATTCATGTTGTGTTCAGTAATTCAGAGA-3′
BW primer:
(SEQ ID NO: 11)
3′-GCGGATCCTTAGATGAGAGTCTTTTCCA-3′

Next, the gene is digested with restriction enzymes EcoRI and BamHI, and then ligated into the plasmid pPGKXDH (Journal of biotechnology, Vol 130., 316-319, 2007) which is digested with the same restriction enzyme to construct pPGKXK.

E. pAUR101_XRXDHXK

PGK1 promoter and terminator from S. cerevisiae, and XKS1 gene from S. cerevisiae are amplified using pPGKXK as a template by means of PCR at an optimal annealing temperature of 55° C. The used primers are follows:

FW primer:
(SEQ ID NO: 12)
5′-GACAGGCGCCAAAGATGCCGATTTGGGCGCGAAT-3′
BW primer:
(SEQ ID NO: 13)
5′-TCCCCCCGGGGTCGACCAGCTTTAACGAACGCAG-3′

Next, the gene is digested with restriction enzymes NarI and XmaI, and then ligated into the plasmid pAUR101_XRXDH which is digested with the same restriction enzyme to construct pAUR101_XRXDHXK.

F. pKM316_XRXDHXK

PGK1 promoter and terminator from S. cerevisiae, XYL1 and XYL2 gene from P. stipitis, and XKS1 gene from S. cerevisiae are amplified by means of PCR at an optimal annealing temperature of 55° C. The used primers are follows:

FW primer:
(SEQ ID NO: 14)
5′-GGACTAGTAGCATCTTAGTGAAAAGGGTGG-3′
BW primer:
(SEQ ID NO: 15)
5′-CCGCTCGAGCGTAAGGAGAAAATACCGCATCA-3′

Next, the gene is digested with restriction enzymes SpeI and XhoI, and then ligated into the plasmid pKM316 which is digested with the same restriction enzyme to construct pKM316_XRXDHXK.

G. pKM316_XRXDHXK_URA3

URA3 gene from S. cerevisiae is amplified by means of PCR at an optimal annealing temperature of 55° C. The used primers are follows:

FW primer:
(SEQ ID NO: 16)
5′-TCCCCGCGGTACCACAGCTTTTCAATTCAATTCA-3′
BW primer:
(SEQ ID NO: 17)
5′-TCCCCGCGGTAGGGTAATAACTGATATAATTAAA-3′

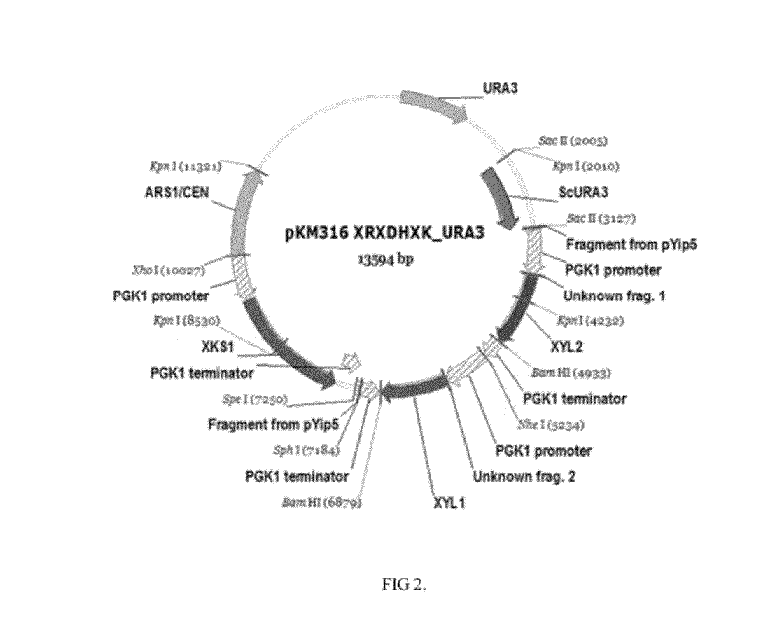

Next, the gene is digested with a restriction enzyme SacII, and then ligated into the plasmid pKM316_XRXDHXK which is digested with the same restriction enzyme to construct pKM316_XRXDHXK_URA3. The pKM316_XRXDHXK_URA3 is shown in FIG. 2.

### Example 3

The following example illustrates the construction of a modified K. marxianus microorganism.

The vectors constructed in Example 2 are introduced into K. marxianus. The expression vector pKM316_XRXDHXK_URA3 is transformed into KM3, KM8 and KM11 by an electroporation method, respectively, and then the production of ethanol is measured with the same method as in Example 1. The result is shown in Table 2.

TABLE 2

| | | | Xylose | Xylitol | Ethanol | | |
|---|---|---|---|---|---|---|---|
| | | ODmax | consumed | produced | produced | Xylitol yield | Ethanol yield |
| Strains | | (g/L) | (g/L) | (g/L) | (g/L) | (g xylitol/g xylose) | (g EtOH/g xylose) |
| KM3 | Control | 8.5 | 24.5 | 13.0 | 0.8 | 0.53 | 0.032 |
| | XR-XDH-XK | 15.3 | 41.0 | 18.5 | 1.8 | 0.45 | 0.044 |
| KM8 | Control | 11.5 | 33.7 | 17.6 | 0.8 | 0.52 | 0.024 |
| | XR-XDH-XK | 14.6 | 36.1 | 14.9 | 2.4 | 0.41 | 0.066 |
| KM11 | Control | 19.2 | 24.4 | 9.9 | 0.2 | 0.40 | 0.009 |
| | XR-XDH-XK | 26.7 | 31.5 | 10.7 | 0.5 | 0.34 | 0.015 |

Referring to Table 2, it can be seen that all modified K. marxianus strains including XR-XDH-XK have enhanced xylose utilization as compared to the precursor strain, and Xylitol yield per gram of xylose is reduced.

Also, it can be seen that the ethanol production of all K. marxianus strains including XR-XDH-XK is about 137%, about 275%, and about 166% greater than that of the precursor strain.

Therefore, K. marxianus strains including XR-XDH-XK which are prepared in the example do not accumulate xylitol in a metabolic pathway of xylose, and productivity of ethanol is enhanced, and thus the ethanol may be industrially used in large quantities.

While example embodiments have been disclosed herein, it should be understood that other variations may be possible. Such variations are not to be regarded as a departure from the spirit and scope of example embodiments of the invention, and all such modifications as would be obvious to one skilled in the art are intended to be included within the scope of the following claims.

What is claimed is:

1. A recombinant microorganism, wherein the microorganism:
converts xylose to xylitol,
converts xylitol to xylulose, and
converts xylulose to xylulose-5-phosphate
and wherein the recombinant microorganism metabolizes xylose to produce a desired chemical.

2. The recombinant microorganism of claim 1, wherein the microorganism exhibits xylose reductase activity, xylitol dehydrogenase activity, and xylulokinase activity.

3. The recombinant microorganism of claim 2, wherein the xylose reductase activity, the xylitol dehydrogenase activity and the xylulokinase activity are heterologous to the microorganism.

4. The recombinant microorganism of claim 3, wherein the xylose reductase activity and the xylitol dehydrogenase activity are derived from Pichia stipitis, and the xylulokinase activity is derived from Saccharomyces cerevisiae.

5. The recombinant microorganism of claim 1, wherein the recombinant microorganism is selected from the group consisting of yeast and bacteria.

6. The recombinant microorganism of claim 5, wherein the recombinant microorganism is selected from the group consisting of Zymomonas, Escherichia, Pseudomonas, Alcaligenes, Salmonella, Shigella, Burkholderia, Oligotropha, Klebsiella, Pichia, Candida, Hansenula, Saccharomyces, Kluyveromyces, Comamonas, Corynebacterium, Brevibacterium, Rhodococcus, Azotobacter, Citrobacter, Enterobacter, Clostridium, Lactobacillus, Aspergillus, Zygosaccharomyces, Dunaliella, Debaryomyces, Mucor, Torulopsis, Methylobacteria, Bacillus, Rhizobium and Streptomyces.

7. The recombinant microorganism of claim 6, wherein the recombinant microorganism is either Kluyveromyces marxianus or Escherichia coli.

8. The recombinant microorganism of claim 1, wherein the desired chemical is an organic acid, an alcohol, an amino acid, or a vitamin.

9. The recombinant microorganism of claim 1, wherein the desired chemical is ethanol.

10. The recombinant microorganism of claim 1, wherein the recombinant microorganism produces the desired chemical at a level greater than the level produced by the precursor microorganism.

11. The recombinant microorganism of claim 1, wherein the recombinant microorganism is a strain deposited under accession number KCTC11951BP, accession number KCTC11952BP, or accession number KCTC11953BP.

12. An expression vector, comprising:
a replication origin;
a promoter;
a nucleotide sequence encoding an enzyme that converts xylose to xylitol, a nucleotide sequence encoding an enzyme that converts xylitol to xylulose, and a nucleotide sequence encoding an enzyme that converts xylulose to xylulose-5-phosphate; and
a terminator.

13. The expression vector of claim 12,
wherein the vector comprises a nucleotide sequence encoding an enzyme represented by SEQ ID NO: 1 or a sequence having at least about 70% sequence homology with the SEQ ID NO: 1,
SEQ ID NO: 2 or a sequence having at least about 70% sequence homology with the SEQ ID NO: 2, and
SEQ ID NO: 3 or a sequence having at least about 70% sequence homology with the SEQ ID NO: 3.

14. The expression vector of claim 12, wherein the replication origin is ARS/CEN (autonomous replication sequence/centromeric sequence).

15. The expression vector of claim 12, wherein the promoter is PGK1 (phosphoglycerate kinase 1) promoter.

16. The expression vector of claim 12, wherein the terminator is CYC1 (cytochrome-c oxidase) terminator.

17. A method of producing a chemical, comprising:
culturing the recombinant microorganism according to claim 1 in a xylose-containing medium, and
recovering the chemical from the medium.

18. The method of claim 17, wherein the xylose is derived from a cellulosic biomass.

19. The method of claim 17, wherein the chemical is an organic acid, alcohol, amino acid or vitamin.

20. The method of claim 17, wherein the chemical is ethanol.

21. The method of claim 17, wherein the recombinant microorganism produces the chemical at a level greater than the level produced by the precursor microorganism.

22. The method claim 21, wherein the recombinant microorganism produces the chemical at a level at least about 130% greater than the level produced by the precursor microorganism.

23. The recombinant microorganism of claim 1, wherein the recombinant microorganism comprises a heterologous nucleic acid encoding xylose reductase, a heterologous nucleic acid encoding xylitol dehydrogenase, and a heterologous nucleic acid encoding xylulokinase.

24. A recombinant microorganism comprising the expression vector of claim 12.

25. A recombinant microorganism comprising the expression vector of claim 13.
